(19) United States
(12) Patent Application Publication
(10) Pub. No.: US 2026/0249291 A1
(43) Pub. Date: Aug. 27, 2026

(54) Methanogen Detection Device and System and Method of Detecting Methanogen

(71) Applicant: Carollo Engineers, Inc., Costa Mesa, CA (US)

(72) Inventors: Vikas Kumar, Vancouver (CA); Hitendra Kumar, Simrol (IN)

(21) Appl. No.: 19/544,799

(22) Filed: Feb. 19, 2026

Related U.S. Application Data
(60) Provisional application No. 63/761,423, filed on Feb. 21, 2025.
(60) Provisional application No. 63/761,454, filed on Feb. 21, 2025.

(51) Int. Cl. B01L 3/00

(52) U.S. Cl. CPC B01L 3/502738

(57) ABSTRACT

A methanogen detection device including a sample inlet, a sample transport channel in fluid communication with the sample inlet, and a capillary flow channel in fluid communication with the sample transport channel. The capillary flow channel is configured to support flow of a sample including methanogens and each of the methanogens includes Cofactor F₄₂₀. The Cofactor F₄₂₀ of each of the methanogens absorbs a first light produced by a light source thereby resulting in excitation and relaxation of the Cofactor F₄₂₀ of each of the methanogens. Excitation and relaxation of the Cofactor F₄₂₀ of each of the methanogens generates a second emission light from the Cofactor F₄₂₀ of each of the methanogens. Also described is a methanogen detection system and method.

### CROSS REFERENCE TO RELATED APPLICATION

This application claims priority to U.S. Provisional Patent Application No. 63/761,454 filed February 21, 2025, and U.S. Provisional Patent Application No. 63/761,423 filed February 21, 2025, the entire contents of each of which are herein incorporated by reference.

### BACKGROUND

### Technical Field

The present disclosure relates to devices, systems, and methods for investigating or analyzing materials by determining their chemical or physical properties or aspects of their composition. In particular, the present disclosure relates to devices, systems, and methods for investigating or analyzing microbial communities within, and/or chemical/physical properties of samples obtained from, wastewater and/or biosolids treatment processes.

### 2. Technical Considerations

The management of wastewater treatment (WWT) and biosolids is an essential aspect of modern sanitation infrastructure, ensuring the safe disposal and reuse of wastewater byproducts. Anaerobic digestion (AD) is a popular method for treating wastewater and biosolids, which utilizes microbial communities to decompose organic matter and produce methane as a valuable renewable energy source. The process of AD relies on the activity of microorganisms, particularly methanogens, which are responsible for converting organic substrates into methane. Methanogens are a class of archaea that have a crucial role in the final stage of AD. They convert volatile fatty acids and hydrogen produced during organic matter degradation into methane gas. Since the abundance of methanogens typically falls within the 1-8% range in most wastewater sludge bioreactors, it represents a bottleneck of AD.

Recent advances in molecular methodologies have greatly increased our knowledge of biological processes, but methanogenic archaea have been largely neglected. Quantitative analysis of methanogens can help maximize process performance, uncover upsets before reactor failure, and ultimately lead to higher resource recovery from the AD systems. Despite the critical role of methanogens, there are significant challenges associated with their monitoring and management.

First, the current molecular instruments and quantitative methods, like DNA sequencing and qPCR (Quantitative Polymerase Chain Reaction) based analysis, demand highly skilled personnel, involve laborious processes, and operate at a slow pace, posing challenges for real-time monitoring. Second, the expense and resource requirements associated with traditional detection methods limit their practicality for routine monitoring in wastewater treatment plants (WWTPs). These methods are expensive, require highly trained personnel, and provide delayed results, making real-time process control difficult. Third, inefficient methanogen monitoring due to delayed results can lead to suboptimal bioreactor analysis, resulting in inviable results which can impact the performances and thereby increase the overall operational costs.

Therefore, there remains a critical need for monitoring and understanding the dynamics of methanogenic populations to optimize AD processes, mitigate process upsets, and maximize biogas production.

### SUMMARY

Accordingly, provided are improved devices, systems, and methods for detecting and analyzing the presence of methanogens in samples obtained from wastewater and/or biosolids treatment processes.

According to some non-limiting embodiments or aspects, provided is a methanogen detection device, including: a sample inlet; a sample transport channel in fluid communication with the sample inlet; and a capillary flow channel in fluid communication with the sample transport channel, the capillary flow channel configured to support flow of a sample including methanogens and each of the methanogens including Cofactor F₄₂₀, where the Cofactor F₄₂₀ of each of the methanogens absorbs a first light produced by a light source thereby resulting in excitation and relaxation of the Cofactor F420 of each of the methanogens, and where excitation and relaxation of the Cofactor F420 of each of the methanogens generates a second emission light from the Cofactor F420 of each of the methanogens.

According to some non-limiting embodiments or aspects, the methanogen detection device may further include a capillary flow medium within the capillary flow channel, the capillary flow medium configured to support the sample thereon.

According to some non-limiting embodiments or aspects, the methanogen detection device may further include an optical filter downstream from the second emission light.

According to some non-limiting embodiments or aspects, the methanogen detection device may further include a transparent top layer, where the sample inlet extends through the transparent top layer; a middle layer, where the middle layer defines the sample transport channel and the capillary flow channel; and a transparent base layer supporting the sample transport channel and the capillary flow channel.

According to some non-limiting embodiments or aspects, the methanogen detection device may further include a sample well extending from the transparent top layer, where the sample well defines the sample inlet.

According to some non-limiting embodiments or aspects, the sample inlet may be configured to receive the sample, and the sample received in the sample inlet flows from the sample inlet to the sample transport channel and from the sample transport channel to the capillary flow channel.

According to some non-limiting embodiments or aspects, the optical filter may be configured to transmit a band of the second emission light including a wavelength range of 470 ± 2.5 nm.

According to some non-limiting embodiments or aspects, a light detector may be configured to detect an intensity of the second emission light transmitted through the optical filter.

According to some non-limiting embodiments or aspects, the sample transport channel may be serpentine.

According to some non-limiting embodiments or aspects, the capillary flow channel may include a cylindrical chamber at a distal portion of the capillary flow channel.

According to some non-limiting embodiments or aspects, the capillary flow medium may be a chromatography paper.

According to some non-limiting embodiments or aspects, the sample well may include a sealing cap.

According to some non-limiting embodiments or aspects, the methanogen detection device may further include a vent in fluid communication with at least one of the following: the sample inlet, the sample transport channel, the capillary flow channel, or any combination thereof.

According to some non-limiting embodiments or aspects, the sample may further include a lysis solution configured to break down a cell wall of each of the methanogens.

According to some non-limiting embodiments or aspects, the methanogen detection device may further include at least one sample analysis channel in fluid communication with the sample transport channel, the at least one sample analysis channel separate from the capillary flow channel, where the at least one sample analysis channel may be configured to test for at least one of the following: pH, alkalinity, ammonia, filamentous bacteria, E. coli, or any combination thereof.

According to some non-limiting embodiments or aspects, provided is a methanogen detection system that may include the methanogen detection device as described above; a light source configured to produce the first light, the light source upstream to the capillary flow channel; an optical filter downstream from the second emission light; and a light detector downstream from the second emission light and the optical filter.

According to some non-limiting embodiments or aspects, the methanogen detection system may further include a capillary flow medium within the capillary flow channel, the capillary flow medium configured to support the sample thereon.

According to some non-limiting embodiments or aspects, the first light produced by the light source may include a wavelength range of 420 ± 2.5 nm.

According to some non-limiting embodiments or aspects, the light detector may include a camera, spectrophotometer, or photodiode.

According to some non-limiting embodiments or aspects, the light detector may be the camera, and the camera may include a smartphone camera.

According to some non-limiting embodiments or aspects, the methanogen detection system may further include a computing device in communication with the light detector, where the computing device may be configured to generate a concentration of the methanogens in the sample based on the intensity of the second emission light detected by the light detector.

According to some non-limiting embodiments or aspects, provided is a methanogen detection system, including: a sample inlet; a sample transport channel in fluid communication with the sample inlet; and a capillary flow channel in fluid communication with the sample transport channel, the capillary flow channel configured to support flow of a sample including methanogens and each of the methanogens including Cofactor F₄₂₀; a light source configured to produce a first light, the light source upstream to the capillary flow channel, where the Cofactor F₄₂₀ of each of the methanogens absorbs the first light produced by the light source thereby resulting in excitation and relaxation of the Cofactor F₄₂₀ of each of the methanogens, and where excitation and relaxation of the Cofactor F₄₂₀ of each of the methanogens generates a second emission light from the Cofactor F₄₂₀ of each of the methanogens; an optical filter downstream from the second emission light, where the optical filter may be configured to transmit a band of the second emission light; a light detector downstream from the second emission light and the optical filter, where the light detector may be configured to detect an intensity of the second emission light transmitted through the optical filter; and a computing device in communication with the light detector, where the computing device may be configured to generate a concentration of the methanogens in the sample based on the intensity of the second emission light detected by the light detector.

According to some non-limiting embodiments or aspects, the methanogen detection system may further include a capillary flow medium within the capillary flow channel, the capillary flow medium configured to support the sample thereon.

According to some non-limiting embodiments or aspects, provided is a methanogen detection method using the methanogen detection device as described above, including: providing a sample to the sample inlet, the sample including methanogens and each of the methanogens including Cofactor F420; irradiating the sample with a first light; detecting an intensity of a second emission light emitted from the Cofactor F420; and generating a concentration of the methanogens in the sample based on the intensity of the second emission light.

According to some non-limiting embodiments or aspects, provided is a methanogen detection method using the methanogen detection system as described above, including: providing a sample to the sample inlet, the sample including methanogens and each of the methanogens including Cofactor F₄₂₀; irradiating the sample with the first light; detecting an intensity of a second emission light emitted from the Cofactor F₄₂₀; and generating a concentration of the methanogens in the sample based on the intensity of the second emission light.

Further non-limiting embodiments or aspects are set forth in the following numbered clauses:

Clause 1: A methanogen detection device, comprising: a sample inlet; a sample transport channel in fluid communication with the sample inlet; and a capillary flow channel in fluid communication with the sample transport channel, the capillary flow channel configured to support flow of a sample comprising methanogens and each of the methanogens comprising Cofactor F₄₂₀, wherein the Cofactor F₄₂₀ of each of the methanogens absorbs a first light produced by a light source thereby resulting in excitation and relaxation of the Cofactor F₄₂₀ of each of the methanogens, and wherein excitation and relaxation of the Cofactor F₄₂₀ of each of the methanogens generates a second emission light from the Cofactor F₄₂₀ of each of the methanogens.

Clause 2: The methanogen detection device of clause 1, further comprising: a capillary flow medium within the capillary flow channel, the capillary flow medium configured to support the sample thereon.

Clause 3: The methanogen detection device of clause 1 or 2, further comprising: an optical filter downstream from the second emission light.

Clause 4. The methanogen detection device of any of clauses 1-3, further comprising: a transparent top layer, wherein the sample inlet extends through the transparent top layer; a middle layer, wherein the middle layer defines the sample transport channel and the capillary flow channel; and a transparent base layer supporting the sample transport channel and the capillary flow channel.

Clause 5: The methanogen detection device of any of clauses 1-4, further comprising: a sample well extending from the transparent top layer, wherein the sample well defines the sample inlet.

Clause 6: The methanogen detection device of any of clauses 1-5, wherein the sample inlet is configured to receive the sample, and wherein the sample received in the sample inlet flows from the sample inlet to the sample transport channel and from the sample transport channel to the capillary flow channel.

Clause 7: The methanogen detection device of any of clauses 1-6, wherein the optical filter is configured to transmit a band of the second emission light comprising a wavelength range of 470 ± 2.5 nm.

Clause 8: The methanogen detection device of any of clauses 1-7, wherein a light detector is configured to detect an intensity of the second emission light transmitted through the optical filter.

Clause 9: The methanogen detection device of any of clauses 1-8, wherein the sample transport channel is serpentine.

Clause 10: The methanogen detection device of any of clauses 1-9, wherein the capillary flow channel comprises a cylindrical chamber at a distal portion of the capillary flow channel.

Clause 11: The methanogen detection device of any of clauses 1-10, wherein the capillary flow medium is chromatography paper.

Clause 12: The methanogen detection device of any of clauses 1-11, wherein the sample well comprises a sealing cap.

Clause 13: The methanogen detection device of any of clauses 1-12, further comprising: a vent in fluid communication with at least one of the following: the sample inlet, the sample transport channel, the capillary flow channel, or any combination thereof.

Clause 14: The methanogen detection device of any of clauses 1-13, wherein the sample further comprises a lysis solution configured to break down a cell wall of each of the methanogens.

Clause 15: The methanogen detection device of any of clauses 1-14, further comprising: at least one sample analysis channel in fluid communication with the sample transport channel, the at least one sample analysis channel separate from the capillary flow channel, wherein the at least one sample analysis channel is configured to test for at least one of the following: pH, alkalinity, ammonia, filamentous bacteria, E. coli, or any combination thereof.

Clause 16: A methanogen detection system, comprising: the methanogen detection device of any of clauses 1-15; a light source configured to produce the first light, the light source upstream to the capillary flow channel; an optical filter downstream from the second emission light; and a light detector downstream from the second emission light and the optical filter.

Clause 17: The methanogen detection system of clause 16, further comprising: a capillary flow medium within the capillary flow channel, the capillary flow medium configured to support the sample thereon.

Clause 18: The methanogen detection system of clause 16 or 17, wherein the first light produced by the light source comprises a wavelength range of 420 ± 2.5 nm.

Clause 19: The methanogen detection system of any of clauses 16-18, wherein the light detector comprises a camera, spectrophotometer, or photodiode.

Clause 20: The methanogen detection system of any of clauses 16-19, wherein the light detector is the camera, and the camera comprises a smartphone camera.

Clause 21: The methanogen detection system of any of clauses 16-20, further comprising: a computing device in communication with the light detector, wherein the computing device is configured to generate a concentration of the methanogens in the sample based on the intensity of the second emission light detected by the light detector.

Clause 22: A methanogen detection system, comprising: a sample inlet; a sample transport channel in fluid communication with the sample inlet; and a capillary flow channel in fluid communication with the sample transport channel, the capillary flow channel configured to support flow of a sample comprising methanogens and each of the methanogens comprising Cofactor F₄₂₀; a light source configured to produce a first light, the light source upstream to the capillary flow channel, wherein the Cofactor F₄₂₀ of each of the methanogens absorbs the first light produced by the light source thereby resulting in excitation and relaxation of the Cofactor F420 of each of the methanogens, and wherein excitation and relaxation of the Cofactor F420 of each of the methanogens generates a second emission light from the Cofactor F420 of each of the methanogens; an optical filter downstream from the second emission light, wherein the optical filter is configured to transmit a band of the second emission light; a light detector downstream from the second emission light and the optical filter, wherein the light detector is configured to detect an intensity of the second emission light transmitted through the optical filter; and a computing device in communication with the light detector, wherein the computing device is configured to generate a concentration of the methanogens in the sample based on the intensity of the second emission light detected by the light detector.

Clause 23: The methanogen detection system of clause 22, further comprising: a capillary flow medium within the capillary flow channel, the capillary flow medium configured to support the sample thereon.

Clause 24: A methanogen detection method using the methanogen detection device of any of clauses 1-15, comprising: providing a sample to the sample inlet, the sample comprising methanogens and each of the methanogens comprising Cofactor F₄₂₀; irradiating the sample with a first light; detecting an intensity of a second emission light emitted from the Cofactor F₄₂₀; and generating a concentration of the methanogens in the sample based on the intensity of the second emission light.

Clause 25: A methanogen detection method using the methanogen detection system of any of clauses 16-23, comprising: providing a sample to the sample inlet, the sample comprising methanogens and each of the methanogens comprising Cofactor F₄₂₀; irradiating the sample with the first light; detecting an intensity of a second emission light emitted from the Cofactor F4₂₀; and generating a concentration of the methanogens in the sample based on the intensity of the second emission light.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter.

### BRIEF DESCRIPTION OF THE DRAWINGS

Additional advantages and details are explained in greater detail below with reference to the non-limiting, exemplary embodiments that are illustrated in the accompanying schematic figures, in which:

FIG. 1 is a perspective, exploded view of a methanogen detection device and system, according to some non-limiting embodiments or aspects of the present disclosure;

FIG. 2 is a perspective view of the methanogen detection device of FIG. 1, according to some non-limiting embodiments or aspects of the present disclosure;

FIG. 3 is a graphical representation showing a relationship between cell dry mass of methanogens and relative fluorescence units (RFU), which can be determined by measuring light emitted from the Cofactor F₄₂₀ of each of the methanogens as shown in FIG. 1; and

FIG. 4 is a schematic diagram of example components of the methanogen detection device and system of FIGS. 1 and 2.

Corresponding reference characters indicate corresponding features throughout the several views of the drawings. The representations set out herein illustrate exemplary aspects of the disclosure, and such representations are not to be construed as limiting the scope of the disclosure in any manner.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. In addition, reference to an action being “based on” a condition may refer to the action being “in response to” the condition. For example, the phrases “based on” and “in response to” may, in some non-limiting embodiments or aspects, refer to a condition for automatically triggering an action (e.g., a specific operation of an electronic device, such as a computing device, a processor, and/or the like).

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, the phrase “in communication” may refer to a relationship capable of reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit.

As used herein, the term “system” may refer to one or more computing devices or combinations of computing devices (e.g., processors, servers, client devices, software applications, components of such, and/or the like). Reference to “a device,” “a server,” “a processor,” and/or the like, as used herein, may refer to a previously-recited device, server, or processor that is recited as performing a previous step or function, a different device, server, or processor, and/or a combination of devices, servers, and/or processors. For example, as used in the specification and the claims, a first device, a first server, or a first processor that is recited as performing a first step or a first function may refer to the same or different device, server, or processor recited as performing a second step or a second function.

While this disclosure is made as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

In some non-limiting embodiments or aspects, the present disclosure proposes microfluidic systems as transformative platforms for transferring complex laboratory analytical procedures onto a single detection device and/or system, which offers distinct advantages such as minimal reagent consumption, enhanced efficiency, and rapid detection. This microfluidic approach presents a straightforward and cost-effective avenue for investigating crucial performance parameters in AD systems. These methods enable the visualization of methanogenic archaea through their intrinsic fluorescent Cofactor F₄₂₀. The present disclosure explores the auto-fluorescence of the methanogens’ Cofactor F₄₂₀ to quantify and enumerate their growth profiles, and the present disclosure provides the capability to study the health of AD systems. Determining the relative abundance of methanogens in engineered anaerobic systems provides crucial onsite results for discovering disturbances before failure or for improvising the overall process.

In some non-limiting embodiments or aspects, analyzing the auto-fluorescent properties of Cofactor F₄₂₀ offers a foundation for the effectiveness of a methanogen detection device 10, system 1, and method of detecting methanogen. The methanogen detection device 10, system 1, and method of detecting methanogen of the present disclosure hold immense potential as tools for scientists and water utilities working to develop more efficient and sustainable treatment processes. The methanogen detection device 10, system 1, and method of detecting methanogen of the present disclosure represent a critical need in the field of wastewater and biosolids treatment for, at least, the following reasons.

Efficiency and Cost-Effectiveness: In some non-limiting embodiments or aspects, the methanogen detection device 10, system 1, and method of detecting methanogen of the present disclosure offer opportunities for rapid, cost-effective, and user-friendly solutions for monitoring methanogen populations in anaerobic digesters, allowing WWTPs to optimize their operations and reduce maintenance costs.

Real-Time Monitoring: In some non-limiting embodiments or aspects, real-time monitoring of methanogens is essential for promptly identifying shifts in microbial communities or process upsets, allowing for immediate corrective actions to be taken to avoid digester failures.

Regulatory Compliance: In some non-limiting embodiments or aspects, numerous regulatory bodies mandate that WWT facilities adhere to specific performance standards, including methane production efficiency. The methanogen detection device 10, system 1, and method of detecting methanogen of the present disclosure facilitate compliance with these regulations, ensuring that facilities meet their environmental obligations.

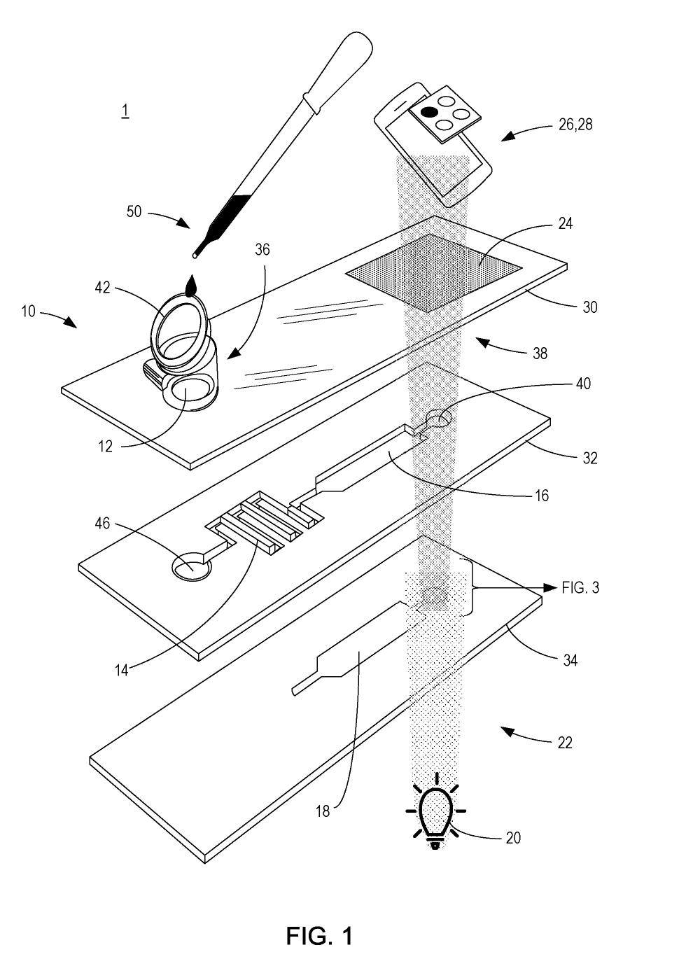

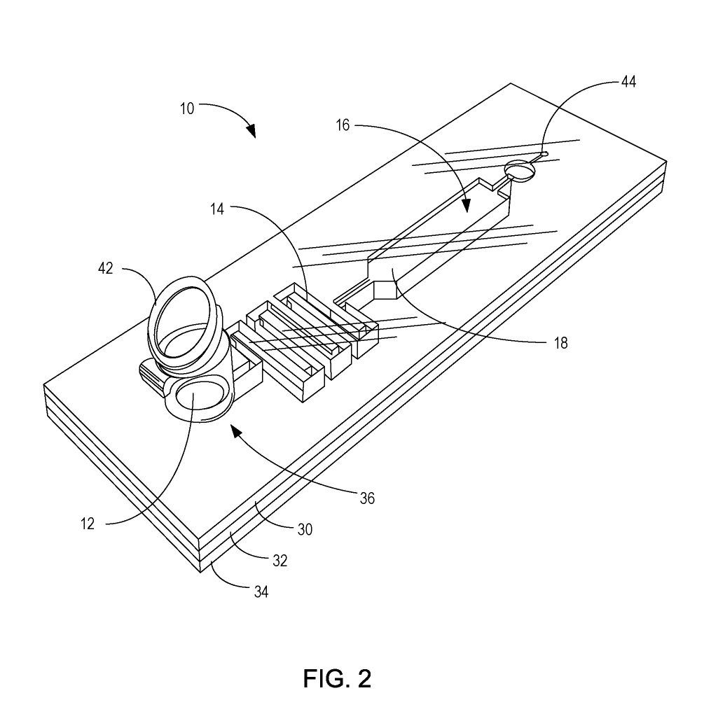

Referring to FIG. 1, in some non-limiting embodiments or aspects, there is shown a perspective, exploded view of a methanogen detection device 10 and system 1 according to some non-limiting embodiments or aspects of the present disclosure. Referring to FIG. 2, there is shown a perspective view of the methanogen detection device 10 of FIG. 1, according to some non-limiting embodiments or aspects of the present disclosure. As shown in FIGS. 1 and 2, the methanogen detection device 10 includes a sample inlet 12, a sample transport channel 14 in fluid communication with the sample inlet 12, and a capillary flow channel 16 in fluid communication with the sample transport channel 14. The capillary flow channel 16 is configured to support flow of a sample 50 having methanogens and each of the methanogens having Cofactor F₄₂₀. The Cofactor F₄₂₀ of each of the methanogens absorbs a first light 22 produced by a light source 20 thereby resulting in excitation and relaxation of the Cofactor F₄₂₀ of each of the methanogens. Excitation and relaxation of the Cofactor F₄₂₀ of each of the methanogens generates a second emission light 38 from the Cofactor F₄₂₀ of each of the methanogens.

In some non-limiting embodiments or aspects, the methanogen detection device 10 can include a capillary flow medium 18 within the capillary flow channel 16. In some non-limiting embodiments or aspects, the capillary flow medium 18 can be chromatography paper. In some non-limiting embodiments or aspects, the capillary flow medium 18 can be a lateral flow device or strip. If the capillary flow medium 18 is a lateral flow device or strip, at least a portion of the lateral flow device or strip can be chromatography paper. The capillary flow medium 18 can be configured to support a sample 50 thereon, and the capillary flow medium 18 can allow capillary flow of the sample 50 thereon/therethrough. The sample 50 can include methanogens and each of the methanogens can include Cofactor F₄₂₀. The sample 50 can be a liquid solution. If the sample 50 is a liquid solution, the sample 50 can include a non-polar organic solvent and a polar solvent, such as water. The sample 50 can also include a lysis solution configured to break down a cell wall of each of the methanogens. The lysis solution may or may not be the non-polar organic solvent. In some non-limiting embodiments or aspects, the lysis solution is chloroform and/or acetone. The capillary flow medium 18 not only allows capillary flow of the sample thereon/therethrough, but can also filter cell debris of the methanogens as a result of the breakdown of the cell wall of each of the methanogens by the lysis solution.

In some non-limiting embodiments or aspects, the sample 50 can include a non-polar organic solvent and a polar solvent, such as water. The sample 50 can flow on/through the capillary flow medium 18 which results in the sample separating into its constituent elements. If the capillary flow medium 18 includes chromatography paper, the capillary flow medium 18 can be made of (at least in part) cellulose, and the cellulose of the chromatography paper attracts the polar solvent, such as water, which results in less mobility of the polar solvent. Therefore, the polar solvent is considered to be the stationary phase and the capillary flow medium 18 is considered to support the stationary phase. The components of the sample 50 that are less polar, including the non-polar organic solvent, are less attracted to the cellulose of the capillary flow medium 18. This results in greater mobility of the non-polar components of the sample and the non-polar organic solvent. Thus, the capillary flow medium 18 allows the sample 50 to be separated into its constituent elements based on polarities of the constituent elements.

In some non-limiting embodiments or aspects, the cell debris of the methanogens, resulting from the breakdown of the cell wall of each of the methanogens by the lysis solution, are retained and immobile on/through the capillary flow medium 18. That is, the capillary flow medium 18 can filter the cell debris of the methanogens. In some non-limiting embodiments or aspects, the Cofactor F₄₂₀ of each of the methanogens is mobile on/through the capillary flow medium 18 and travels to a cylindrical chamber 40 at a distal portion of the capillary flow channel 16.

In some non-limiting embodiments or aspects, the methanogen detection system 1 can include the methanogen detection device 10 as well as the light source 20 configured to produce the first light 22. The light source 20 can be upstream to the capillary flow channel 16 and the capillary flow medium 18 when present within the capillary flow channel 16. In some non-limiting embodiments or aspects, the light source 20 can be a light-emitting diode (LED). The first light 22 produced by the light source 20 can have, at least, a wavelength range of 420 ± 2.5 nm. In other embodiments, the first light 22 produced by the light source 20 can have various wavelengths, in which one of the wavelengths is approximately 420 nm. The light source 20 can be a separate device from the methanogen detection device 10 or it can be integrated into themethanogen detection device 10.

In some non-limiting embodiments or aspects, the Cofactor F₄₂₀ of each of the methanogens absorbs the first light 22 produced by the light source 20 thereby resulting in excitation and relaxation of the Cofactor F₄₂₀ of each of the methanogens. In particular, the Cofactor F₄₂₀ of each of the methanogens absorbs the wavelength of approximately 420 nm of the first light 22 produced by the light source 20 thereby resulting in excitation and relaxation of the Cofactor F₄₂₀ of each of the methanogens. Excitation and relaxation of the Cofactor F₄₂₀ of each of the methanogens generates a second emission light 38 from the Cofactor F₄₂₀ of each of the methanogens. The second emission light 38 generated by excitation and relaxation of the Cofactor F₄₂₀ can have a wavelength range of 470 ± 2.5 nm. Furthermore, the second emission light 38 generated by excitation and relaxation of the Cofactor F₄₂₀ can have various wavelengths, in which one of the wavelengths is approximately470 nm.

In some non-limiting embodiments or aspects, the above-described process of absorbance of the first light 22 produced by the light source 20, excitation and relaxation, and emittance of the second emission light 38 generated by the excitation and relaxation of the Cofactor F₄₂₀ generally illustrates the process of fluorescence of the Cofactor F₄₂₀ of each of the methanogens. Fluorescence of Cofactor F₄₂₀ occurs through the following specific process. The Cofactor F₄₂₀ molecules of each of the methanogens absorb the first light 22 produced by the light source 20 thereby resulting in excitation of electrons of the Cofactor F₄₂₀ molecules of each of the methanogens. In particular, the Cofactor F₄₂₀ molecules of each of the methanogens absorb the wavelength of approximately 420 nm of the first light 22 produced by the light source 20 thereby resulting in excitation of electrons of the Cofactor F₄₂₀ molecules of each of the methanogens. Excitation of the electrons of the Cofactor F₄₂₀ molecules of each of the methanogens results in the electrons transitioning to a higher energy state. When the electrons of the Cofactor F₄₂₀ molecules relax or return to a lower energy state, the electrons of the Cofactor F420 molecules emit photons at a longer wavelength than the wavelength of approximately 420 nm of the first light 22. The emitted photons make up or constitute the second emission light 38 from the Cofactor F420 of each of the methanogens, which is observable as fluorescence. The second emission light 38 generated by the emitted photons from the electrons of the Cofactor F420 molecules relaxing or returning to a lower energy state can have a wavelength range of 470 ± 2.5 nm. Furthermore, the second emission light 38 generated by excitation and relaxation of the Cofactor F420 can have various wavelengths, in which one of the wavelengths is approximately 470 nm.

In some non-limiting embodiments or aspects, the methanogen detection device 1 can include an optical filter 24 downstream from the second emission light 38 (e.g., between the second emission light 38 and the detector 26). The optical filter 24 can be configured to transmit a band of the second emission light 38. The band of the second emission light 38 transmitted by the optical filter 24 can have a wavelength range of 470 ± 2.5 nm. In some non-limiting embodiments or aspects, the optical filter 24 is a band-pass optical filter.

In some non-limiting embodiments or aspects, the methanogen detection system 1 can include a light detector 26 downstream from the second emission light 38 and the optical filter 24. The light detector 26 may be a separate device from the methanogen detection device 10 or it may be incorporated into the methanogen detection device 10. The light detector 26 can be configured to detect an intensity of the second emission light 38 transmitted through the optical filter 24. The light detector 26 can be a spectrophotometer, camera, or photodiode. If the light detector 26 is a camera, the camera can be a smartphone camera. If the light detector 26 is a spectrophotometer, the spectrophotometer can be a handheld spectrophotometer. If the light detector 26 is a photodiode, the photodiode can be a photodiode sensor. Using readily available equipment, such as a spectrophotometer, camera, or photodiode for the light detector 26, makes the methanogen detection device 10, system 1, and method of detecting methanogen accessible and cost-effective.

In some non-limiting embodiments or aspects, the methanogen detection system 1 can include a computing device 28. The computing device 28 may be a separate device from the methanogen detection device 10 or it may be incorporated into themethanogen detection device 10. The computing device 28 can be configured to generate a concentration of the methanogens in the sample based on the intensity of the second emission light 38 detected by the light detector 26. The computing device 28 can be one or more electronic device(s) that is/are configured to process data. Each computing device 28 can include a processor, a user interface, an input device, a display, a memory, a network interface, etc. That is, each computing device 28 can include any components necessary to receive, store, process, and/or output data. in some non-limiting embodiments or aspects, the light detector 26 is in communication with the computing device 28. In some non-limiting embodiments or aspects, the computing device 28 is operatively connected to and in communication with at least one of the following: the light detector 26, the light source 20, or any combination thereof.

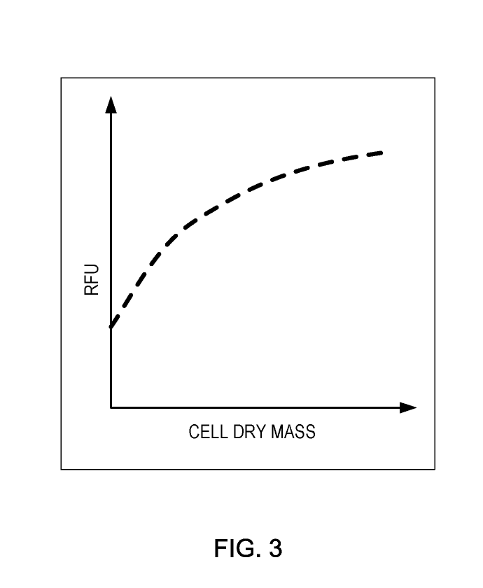

In some non-limiting embodiments or aspects, if the methanogen detection system 1 includes a computing device 28 in communication with the light detector 26, such as a smartphone having a camera as shown in FIG. 1, the second emission light 38 transmitted through the optical filter 24 can be recorded and stored in a memory of the computing device 28, such as in the form of an image. The intensity of the second emission light 38 transmitted through the optical filter 24, as the fluorescence signal in this image, can be used to determine the concentration of the methanogens in the sample 50. As shown in FIG. 3, a calibration curve can be established using known concentrations of methanogens (based on cell dry mass of the methanogens) in various samples and measuring the intensity of the light transmitted through the optical filter 24 (as relative fluorescence units (RFU)) for each of the known concentrations of the methanogens in the various samples. In some non-limiting embodiments or aspects, the calibration curve can be stored in the memory or storage of the computing device 28. In some non-limiting embodiments or aspects, the calibration curve can be incorporated into a computer program product including program instructions that can be executed by a processor of the computing device 28. The computer program product can be a mobile app or web app and can utilize an R software package. For a sample 50 having an unknown concentration of methanogens, the computing device 28 can apply the calibration curve to the second emission light 38 transmitted through the optical filter 24, and recorded and stored in a memory of the computing device 28, such as in the form of an image, and can determine the concentration of the methanogens in the sample 50.

In some non-limiting embodiments or aspects, the intensity of the second emission light 38 transmitted through the optical filter 24, such as the fluorescence signal in an image, can be used to determine the concentration of the methanogens in the sample 50. In other words, the unknown methanogen concentration in the sample 50 will be calibrated with the known methanogen concentrations based on corresponding emission signals. A computer software or smartphone application can be used to transcribe the image intensity to the corresponding numerical values for concentration. Unknown concentrations within samples 50 can be examined using the calibration curve to determine the relative methanogen concentrations in the samples 50.

Referring to FIG. 1, in some non-limiting embodiments or aspects, the methanogen detection device 10 can include a transparent top layer 30, a middle layer 32, and transparent base layer 34. The sample inlet 12 can extend through the transparent top layer 30. The middle layer 32 can define the sample transport channel 14 and the capillary flow channel 16. The transparent base layer 34 can support the sample transport channel 14, the capillary flow channel 16, and the capillary flow medium 18 when present within the capillary flow channel 16. If the methanogen detection device 10 includes a transparent top layer 30, a middle layer 32, and transparent base layer 34, the light source 20 can be positioned below the transparent base layer 34, the optical filter can be positioned above or atop the transparent top layer 30, and the capillary flow medium 18 can be positioned atop the transparent base layer 34. Each of the transparent top layer 30, the middle layer 32, and the transparent base layer 34 can have dimensions of 75 mm x 25 mm. In some non-limiting embodiments or aspects, the transparent base layer 34 is a standard microscopic glass slide having dimensions of 75 mm x 25 mm. In some non-limiting embodiments or aspects, the middle layer 32 is a 1 mm thick acrylic sheet. In some non-limiting embodiments or aspects, the transparent top layer 30 is a 1 mm thick acrylic sheet.

In some non-limiting embodiments or aspects, the methanogen detection device 10 can include a sample well 36 extending from the transparent top layer 30. The sample well 36 can define the sample inlet 12. The sample well 36 can have a tubular structure extending from the transparent top layer 30. The sample inlet 12 and the sample well 36, when present, are configured to receive the sample 50. With the sample 50 received in the sample inlet 12 and the sample well 36, when present, the sample 50 can flow from the sample inlet 12 to the sample transport channel 14 and from the sample transport channel 14 to the capillary flow channel 16 or onto/through the capillary flow medium 18 within the capillary flow channel 16, when the capillary flow medium 18 is present within the capillary flow channel 16. The sample well 36 can have a sealing cap 42 that is configured to seal the sample inlet 12 and the sample well 36 from an external atmosphere.

In some non-limiting embodiments or aspects, the methanogen detection device 10 can include a sample inlet reservoir 46. The sample inlet reservoir 46 can be in fluid communication with the sample transport channel 14. A user of the methanogen detection device 10 can drop a sample 50 in the sample inlet 12. The sample 50 is then received by the sample inlet reservoir 46 and proceeds to flow through the sample transport channel 14. Flow of the sample 50 through the sample transport channel 14 can be aided at least in part by capillary action. In some non-limiting embodiments or aspects, the sample transport channel 14 can have a channel width of 100-1,000 µm. The sample can also be pushed from the sample inlet reservoir 46 to force it to flow through the sample transport channel 14.

In some non-limiting embodiments or aspects, the sample transport channel 14 can be serpentine and/or meandering. As described previously, the sample 50 can also include a lysis solution configured to break down a cell wall of each of the methanogens. In some non-limiting embodiments or aspects, the lysis solution is chloroform and/or acetone. When the sample transport channel 14 is serpentine and/or meandering, the sample 50 flows through the serpentine and/or meandering sample transport channel 14 in sufficient time to allow the lysis solution to break down the cell wall of each of the methanogens. Using a lysis solution to break down the cell wall of each of the methanogens allows intracellular Cofactor F₄₂₀ of each of the methanogens of the sample 50 to release to the mobile solution and move on/through the capillary flow medium 18 and travel to a cylindrical chamber 40 at a distal portion of the capillary flow channel 16. As identified above, the auto-fluorescence property of Cofactor F₄₂₀ generates emission of photons when incident light having a wavelength of 420 nm is absorbed by the Cofactor F420 molecules.

In some non-limiting embodiments or aspects, after flow through the sample transport channel 14, the sample 50 proceeds to flow to the capillary flow channel 16. When the capillary flow medium 18 is present within the capillary flow channel 16, the sample 50 can flow from the sample transport channel 14 onto/through the capillary flow medium 18 within the capillary flow channel 16. In some non-limiting embodiments or aspects, the width of the capillary flow channel 16 is larger than the width of the sample transport channel 14. The capillary flow channel 16 can have a cylindrical chamber 40 (or other cross-sectional shape) at a distal portion of the capillary flow channel 16. The capillary flow medium 18 can have a shape that matches the profile of the capillary flow channel 16. For example, when the capillary flow channel 16 has a cylindrical chamber 40 at a distal portion thereof, the capillary flow medium 18 can have a disk-shaped end that fits securely into the cylindrical chamber 40. In some non-limiting embodiments or aspects, the cylindrical chamber 40 does not include the capillary flow medium 18. The cylindrical chamber 40 allows a sufficient volume (e.g., 25-500 µL) of the sample 50 and a sufficient amount of the Cofactor F₄₂₀ of each of the methanogens within the sample 50 to be present in the cylindrical chamber 40 for exposure of the Cofactor F₄₂₀ of each of the methanogens to the first light 22 from the light source 20.

In some non-limiting embodiments or aspects, flow of the sample 50 through the sample transport channel 14 can be aided at least in part by a vent 44 in fluid communication with at least one of the following: the sample inlet 12, the sample transport channel 14, the capillary flow channel 16, or any combination thereof. Referring to FIG. 2, the transparent top layer 30 can define the vent 44 and the vent 44 can be in fluid communication with the capillary flow channel 16. The vent 44 can be a hole serving as a vent for the sample transport channel 14 and the capillary flow channel 16, each of which can be defined by the middle layer 32. The hole can be a 1 mm diameter hole. The vent 44 allows air to escape from the sample transport channel 14 and the capillary flow channel 16 while the sample 50 flows through the methanogen detection device 10.

In some non-limiting embodiments or aspects, while not shown in the Figures, the methanogen detection device 10 can include at least one sample analysis channel in fluid communication with the sample transport channel 14. The at least one sample analysis channel can be a separate and distinct channel from the capillary flow channel 16. The at least one sample analysis channel can include the capillary flow medium 18 within the sample analysis channel. The capillary flow medium 18 can be configured to support the sample thereon/therethrough. The at least one sample analysis channel can be configured to test for at least one of the following: pH, alkalinity, ammonia, filamentous bacteria, E. coli, or any combination thereof. For example, each of the at least one sample analysis channel can include a well or reservoir at a distal potion thereof. The reservoir/well of each of the at least one sample analysis channel can include different sensors, such as color-based pH detectors (e.g., Phenolphthalein), specific reagents that interact with specific bacterium types (e.g., nucleic acid-based biomarkers), and different excitation lights (e.g., LEDs) placed upstream from the reservoir/well and optical filter combinations for different wavelengths placed downstream from the reservoir/well. By adding supplemental reservoirs and/or chemical wells, the construction of the detection device 10 can be modified to allow for multiple detections to be made from the same sample 50 and detection device 10.

In some non-limiting embodiments or aspects, the transparent top layer 30 and the middle layer 32 can be fabricated using a stereolithography 3D printer or a laser cutter. Assembly of the transparent top layer 30, the middle layer 32, and the transparent base layer 34 can be facilitated by a double-sided acrylic adhesive.

In some non-limiting embodiments or aspects, as discussed above, the present disclosure is also directed to a methanogen detection system 1. The methanogen detection system 1 can include the methanogen detection device 10 including the sample inlet 12, the sample transport channel 14, the capillary flow channel 16, and the optical filter 24, and can also include the light source 20, the light detector 26, and a computing device 28. The sample transport channel 14 can be in fluid communication with the sample inlet 12. The capillary flow channel 16 can be in fluid communication with the sample transport channel 14. The capillary flow channel 16 can be configured to support flow of the sample 50 having methanogens and each of the methanogens having Cofactor F₄₂₀. The light source 20 can be configured to produce a first light 22, and the light source 20 can be upstream to the capillary flow channel 16. The Cofactor F₄₂₀ of each of the methanogens can absorb the first light 22 produced by the light source 20 thereby resulting in excitation and relaxation of the Cofactor F420 of each of the methanogens. Excitation and relaxation of the Cofactor F420 of each of the methanogens can generate a second emission light 38 from the Cofactor F420 of each of the methanogens. The optical filter 24 can be downstream from the second emission light 38, and the optical filter 24 can be configured to transmit a band of the second emission light 38 having a wavelength range of 470 ± 2.5 nm. The light detector 26 can be downstream from the second emission light 38 and the optical filter 24, and the light detector 26 can be configured to detect an intensity of the second emission light 38 transmitted through the optical filter 24. The computing device 28 can be in communication with the light detector 26, and the computing device 28 can be configured to generate a concentration of the methanogens in the sample 50 based on the intensity of the second emission light 38 detected by the light detector 26. The methanogen detection system can include the capillary flow medium 18 within the capillary flow channel 16, and the capillary flow medium 18 can be configured to support the sample 50 thereon.

Referring to FIG. 1, in some non-limiting embodiments or aspects, the computing device 28 can be a smartphone, which can include, for example, a processor, a user interface, an input device, a display, a memory, a network interface, etc. As described above and throughout this disclosure, the methanogen detection system 1 can include a light detector 26 downstream from the second emission light 38 and the optical filter 24. The light detector 26 can be configured to detect an intensity of the second emission light 38 transmitted through the optical filter 24. The light detector 26 can be a spectrophotometer or a camera. If the light detector 26 is a camera, spectrophotometer, or photodiode, the computing device 28 can be any computing device that is capable of communicating with the camera, spectrophotometer, or photodiode; receiving data from the camera, spectrophotometer, or photodiode; storing data, such as data received from the camera, spectrophotometer, or photodiode; processing data, such as data received from the camera, spectrophotometer, or photodiode; and/or outputting data. Specifically, if the light detector 26 is a camera, spectrophotometer, or photodiode, the computing device 28 can be any computing device that is capable of communicating with the camera, spectrophotometer, or photodiode, receiving the intensity of the second emission light 38 transmitted through the optical filter 24, storing the intensity of the second emission light 38 transmitted through the optical filter 24, and outputting a concentration of the methanogens in the sample 50 using the calibration curve. In some non-limiting embodiments or aspects, the light detector 26 is a camera, and the camera can be a smartphone camera and the computing device 28 can be a smartphone. Using readily available equipment, such as smartphone for the computing device 28, makes the methanogen detection device 10, system 1, and method of detecting methanogen accessible and cost-effective.

In some non-limiting embodiments or aspects, more generally, the computing device 28 can be any computing device that is capable of controlling and/or communicating with any of the light source 20 and the light detector 26; receiving data from any of the light source 20 and the light detector 26; storing data, such as data received from any of the light source 20 and the light detector 26; processing data, such as data received from any of the light source 20 and the light detector 26; and/or outputting data. Specifically, the computing device 28 can be any computing device that is capable of operating the light source 20 and the light detector 26; communicating with the light source 20 and the light detector 26; receiving the intensity of the second emission light 38 transmitted through the optical filter 24 from the light detector 26; storing the intensity of the second emission light 38 transmitted through the optical filter 24 and received from the light detector 26; processing the intensity of the second emission light 38 transmitted through the optical filter 24 and received from the light detector 26; and outputting a concentration of the methanogens in the sample using the calibration curve.

In some non-limiting embodiments or aspects, the methanogen detection system 1 can have any of the same features as the methanogen detection device 10 described above and throughout this disclosure. In some non-limiting embodiments or aspects, the methanogen detection system 1 can have more features than the methanogen detection device 10 described above and throughout this disclosure.

Referring to FIG. 3, in some non-limiting embodiments or aspects, there is shown a graphical representation showing a relationship between cell dry mass of methanogens and relative fluorescence units (RFU), which can be determined by measuring the second emission light 38 emitted from the Cofactor F₄₂₀ of each of the methanogens as shown in FIG. 1. In some non-limiting embodiments or aspects, a standard curve can be created by correlating the Cofactor F420 concentration with relative fluorescent units (RFU) values. This standard curve can be incorporated into a computer program product including program instructions that can be executed by the computing device 28. As discussed above, the computing device 28 can be configured to generate a concentration of the methanogens in the sample 50 based on the intensity of the second emission light 38 detected by the light detector 26. The computer program product can be a mobile app or web app and can utilize an R software package. In some non-limiting embodiments or aspects, the concentration of the Cofactor F420 relative to the methanogen concentration in the sample can be determined using reference values.

In some non-limiting embodiments or aspects, the present disclosure is also directed to a method of detecting methanogen. The method of detecting methanogen can include irradiating a sample comprising methanogens and each of the methanogens comprising Cofactor F₄₂₀ with a first light, detecting an intensity of a second emission light emitted from the Cofactor F₄₂₀ of each of the methanogens, and generating a concentration of the methanogens in the sample based on the intensity of the second emission light.

In some non-limiting embodiments or aspects, the method of detecting methanogen can include any of the following elements: adding a lysis solution to the sample to break down a cell wall of each of the methanogens; contacting the sample to a sample transport channel; contacting the sample to a capillary flow medium; filtering the second emission light through an optical filter by transmitting a band of the second emission light having a wavelength range of 470 ± 2.5 nm; and/or analyzing the sample for at least one of the following: pH, alkalinity, ammonia, filamentous bacteria, E. coli, or any combination thereof.

In some non-limiting embodiments or aspects, the methanogen detection device 10, system 1, and method of detecting methanogen each provide the following benefits. Each of the detection device 10, system 1, and method: provides guidance to utilities by offering utilities comprehensive insights into biosolid treatment, regulatory perspectives, and advanced removal technologies; facilitates informed decision-making by keeping utilities well-informed with the latest developments in AD and methanogen detection; generates proactive engagement in methanogen detection and biosolid treatment initiatives; offers solutions in methanogen detection, aligning with industry demands and trends; develops and standardizes tools and databases to support engineers as regulatory frameworks evolve; ensures readiness in navigating regulatory changes and provides users with valuable insights; extends support to users in anaerobic bioreactor and biosolid applied research; and/or actively allows for collaboration with utilities to tailor research efforts, fostering innovation and advancing biosolid treatment practices.

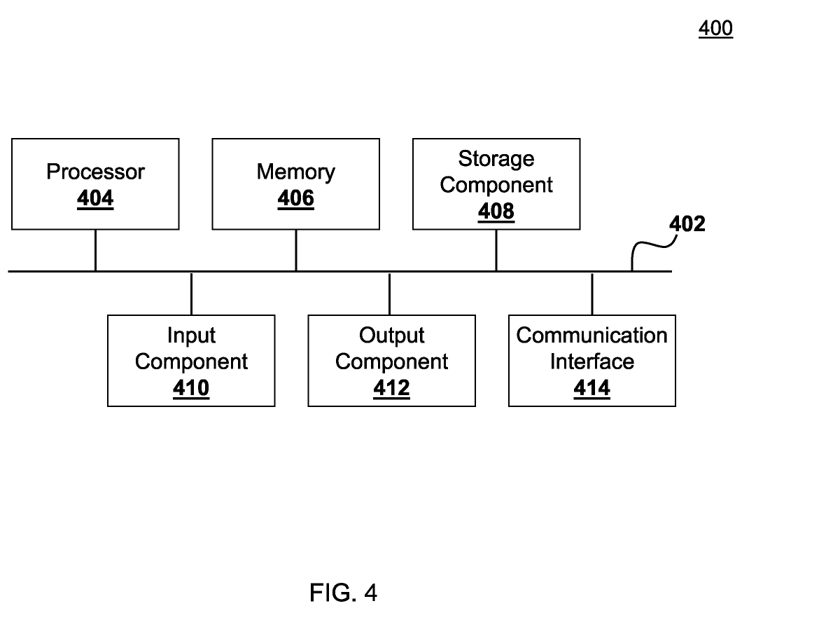

Referring now to FIG. 4, shown is a diagram of example components of device 400, according to non-limiting embodiments or aspects. Device 400 may correspond to at least one of the methanogen detection system 1, the methanogen detection device 10, the light detector 26, and/or the computing device 28 in FIGS. 1 and 2. In some non-limiting embodiments or aspects, such systems or devices in FIGS. 1-3 may include at least one device 400 and/or at least one component of device 400. The number and arrangement of components shown in FIG. 4 are provided as an example. In some non-limiting embodiments or aspects, device 400 may include additional components, fewer components, different components, or differently arranged components than those shown in FIG. 4. Additionally or alternatively, a set of components (e.g., one or more components) of device 400 may perform one or more functions described as being performed by another set of components of device 400.

As shown in FIG. 4, device 400 may include bus 402, processor 404, memory 406, storage component 408, input component 410, output component 412, and communication interface 414. Bus 402 may include a component that permits communication among the components of device 400. In some non-limiting embodiments or aspects, processor 404 may be implemented in hardware, firmware, or a combination of hardware and software. For example, processor 404 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 406 may include random access memory (RAM), read only memory (ROM), and/or another type of dynamic or static storage device (e.g., flash memory, magnetic memory, optical memory, etc.) that stores information and/or instructions for use by processor 404.

With continued reference to FIG. 4, storage component 408 may store information and/or software related to the operation and use of device 400. For example, storage component 408 may include a hard disk (e.g., a magnetic disk, an optical disk, a magneto-optic disk, a solid-state disk, etc.) and/or another type of computer-readable medium. Input component 410 may include a component that permits device 400 to receive information, such as via user input (e.g., a touch screen display, a keyboard, a keypad, a mouse, a button, a switch, a microphone, etc.). Additionally or alternatively, input component 410 may include a sensor for sensing information (e.g., a global positioning system (GPS) component, an accelerometer, a gyroscope, an actuator, etc.). Output component 412 may include a component that provides output information from device 400 (e.g., a display, a speaker, one or more light-emitting diodes (LEDs), etc.). Communication interface 414 may include a transceiver-like component (e.g., a transceiver, a separate receiver and transmitter, etc.) that enables device 400 to communicate with other devices, such as via a wired connection, a wireless connection, or a combination of wired and wireless connections. Communication interface 414 may permit device 400 to receive information from another device and/or provide information to another device. For example, communication interface 414 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 400 may perform one or more processes described herein. Device 400 may perform these processes based on processor 404 executing software instructions stored by a computer-readable medium, such as memory 406 and/or storage component 408. A computer-readable medium may include any non-transitory memory device. A memory device may include memory space located inside of a single physical storage device or memory space spread across multiple physical storage devices. Software instructions may be read into memory 406 and/or storage component 408 from another computer-readable medium or from another device via communication interface 414. When executed, software instructions stored in memory 406 and/or storage component 408 may cause processor 404 to perform one or more processes described herein. Additionally or alternatively, hardwired circuitry may be used in place of or in combination with software instructions to perform one or more processes described herein. Thus, embodiments described herein are not limited to any specific combination of hardware circuitry and software. The term “configured to,” as used herein, may refer to an arrangement of software, device(s), and/or hardware for performing and/or enabling one or more functions (e.g., actions, processes, steps of a process, and/or the like). For example, “a processor configured to” may refer to a processor that executes software instructions (e.g., program code) that cause the processor to perform one or more functions.

While exemplary designs have been described above in the detailed description, those of ordinary skill in the art will understand that the exemplary designs of the present disclosure can be further modified within the spirit and scope of this disclosure. Therefore, the above-described exemplary designs should not be considered to limit the scope of the appended claims.

What is claimed is:

1. A methanogen detection device, comprising:
a sample inlet;
a sample transport channel in fluid communication with the sample inlet; and
a capillary flow channel in fluid communication with the sample transport channel, the capillary flow channel configured to support flow of a sample comprising methanogens and each of the methanogens comprising Cofactor F₄₂₀
wherein the Cofactor F₄₂₀ of each of the methanogens absorbs a first light produced by a light source thereby resulting in excitation and relaxation of the Cofactor F₄₂₀ of each of the methanogens, and
wherein excitation and relaxation of the Cofactor F₄₂₀ of each of the methanogens generates a second emission light from the Cofactor F₄₂₀ of each of the methanogens.

2. The methanogen detection device of claim 1, further comprising:
a capillary flow medium within the capillary flow channel, the capillary flow medium configured to support the sample thereon.

3. The methanogen detection device of claim 1, further comprising:
an optical filter downstream from the second emission light.

4. The methanogen detection device of claim 1, further comprising:
a transparent top layer, wherein the sample inlet extends through the transparent top layer;
a middle layer, wherein the middle layer defines the sample transport channel and the capillary flow channel; and
a transparent base layer supporting the sample transport channel and the capillary flow channel.

5. The methanogen detection device of claim 4, further comprising:
a sample well extending from the transparent top layer, wherein the sample well defines the sample inlet.

6. The methanogen detection device of claim 1, wherein the sample inlet is configured to receive the sample, and
wherein the sample received in the sample inlet flows from the sample inlet to the sample transport channel and from the sample transport channel to the capillary flow channel.

7. The methanogen detection device of claim 3, wherein the optical filter is configured to transmit a band of the second emission light comprising a wavelength range of 470 ± 2.5 nm.

8. The methanogen detection device of claim 3, wherein a light detector is configured to detect an intensity of the second emission light transmitted through the optical filter.

9. The methanogen detection device of claim 1, wherein the sample transport channel is serpentine.

10. The methanogen detection device of claim 1, wherein the capillary flow channel comprises a cylindrical chamber at a distal portion of the capillary flow channel.

11. The methanogen detection device of claim 2, wherein the capillary flow medium is chromatography paper.

12. The methanogen detection device of claim 5, wherein the sample well comprises a sealing cap.

13. The methanogen detection device of claim 1, further comprising:
a vent in fluid communication with at least one of the following: the sample inlet, the sample transport channel, the capillary flow channel, or any combination thereof.

14. The methanogen detection device of claim 1, wherein the sample further comprises a lysis solution configured to break down a cell wall of each of the methanogens.

15. The methanogen detection device of claim 1, further comprising:
at least one sample analysis channel in fluid communication with the sample transport channel, the at least one sample analysis channel separate from the capillary flow channel,
wherein the at least one sample analysis channel is configured to test for at least one of the following: pH, alkalinity, ammonia, filamentous bacteria, E. coli or any combination thereof.

16. A methanogen detection system, comprising:
the methanogen detection device of claim 1;
a light source configured to produce the first light, the light source upstream to the capillary flow channel;
an optical filter downstream from the second emission light; and
a light detector downstream from the second emission light and the optical filter.

17. The methanogen detection system of claim 16, further comprising:
a capillary flow medium within the capillary flow channel, the capillary flow medium configured to support the sample thereon.

18. The methanogen detection system of claim 16, wherein the first light produced by the light source comprises a wavelength range of 420 ± 2.5 nm.

19. The methanogen detection system of claim 16, wherein the light detector comprises a camera, spectrophotometer, or photodiode.

20. The methanogen detection system of claim 19, wherein the light detector is the camera, and the camera comprises a smartphone camera.

21. The methanogen detection system of claim 16, further comprising:
a computing device in communication with the light detector, wherein the computing device is configured to generate a concentration of the methanogens in the sample based on the intensity of the second emission light detected by the light detector.

22. A methanogen detection system, comprising:
a sample inlet;
a sample transport channel in fluid communication with the sample inlet;
a capillary flow channel in fluid communication with the sample transport channel, the capillary flow channel configured to support flow of a sample comprising methanogens and each of the methanogens comprising Cofactor F₄₂₀;
a light source configured to produce a first light, the light source upstream to the capillary flow channel,
wherein the Cofactor F₄₂₀ of each of the methanogens absorbs the first light produced by the light source thereby resulting in excitation and relaxation of the Cofactor F₄₂₀ of each of the methanogens, and
wherein excitation and relaxation of the Cofactor F₄₂₀ of each of the methanogens generates a second emission light from the Cofactor F₄₂₀ of each of the methanogens;
an optical filter downstream from the second emission light, wherein the optical filter is configured to transmit a band of the second emission light;
a light detector downstream from the second emission light and the optical filter, wherein the light detector is configured to detect an intensity of the second emission light transmitted through the optical filter; and
a computing device in communication with the light detector, wherein the computing device is configured to generate a concentration of the methanogens in the sample based on the intensity of the second emission light detected by the light detector.

23. The methanogen detection system of claim 22, further comprising:
a capillary flow medium within the capillary flow channel, the capillary flow medium configured to support the sample thereon.

24. A methanogen detection method using the methanogen detection device of claim 1, comprising:
providing a sample to the sample inlet, the sample comprising methanogens and each of the methanogens comprising Cofactor F₄₂₀;
irradiating the sample with a first light;
detecting an intensity of a second emission light emitted from the Cofactor F₄₂₀; and
generating a concentration of the methanogens in the sample based on the intensity of the second emission light.

25. A methanogen detection method using the methanogen detection system of claim 16, comprising:
providing a sample to the sample inlet, the sample comprising methanogens and each of the methanogens comprising Cofactor F₄₂₀;
irradiating the sample with the first light;
detecting an intensity of a second emission light emitted from the Cofactor F₄₂₀; and
generating a concentration of the methanogens in the sample based on the intensity of the second emission light.
